(19) United States
(12) Patent Application Publication
(10) Pub. No.: US 2019/0053851 A1
(43) Pub. Date: Feb. 21, 2019

(54) SURGICAL NAVIGATION SYSTEM AND METHOD FOR PROVIDING AN AUGMENTED REALITY IMAGE DURING OPERATION

(71) Applicant: Holo Surgical Inc., Chicago, IL (US)

(72) Inventors: Kris B. Siemionow, Chicago, IL (US); Cristian J. Luciano, Evergreen Park, IL (US)

(21) Appl. No.: 16/101,459

(22) Filed: Aug. 12, 2018

(30) Foreign Application Priority Data
Aug. 15, 2017 (EP) 17186306.1

(51) Int. Cl. A61B 34/10; G06T 7/10; A61B 34/20

(52) U.S. Cl. CPC A61B 34/10; G06T 19/003; A61B 34/20; G06T 7/10

(57) ABSTRACT

A surgical navigation system includes: a 3D display with a see-through mirror that is partially transparent and partially reflective; a tracking system comprising means for real-time tracking of: a surgeon's head, the see-through mirror and a patient anatomy to provide current position and orientation data; a source of a patient anatomy data; a surgical navigation image for generating a surgical navigation image comprising at least the patient anatomy data, in accordance with the current position and orientation data provided by the tracking system based on the current relative position and orientation of the surgeon's head, the see-through mirror and the patient anatomy; the 3D display configured to emit the surgical navigation image towards the see-through mirror, such that an augmented reality image collocated with the patient anatomy in the surgical field underneath the see-through mirror is visible to a viewer looking from above the see-through mirror towards the surgical field.

### TECHNICAL FIELD

The present disclosure relates to surgical navigation systems, in particular to a system and method for operative planning and execution of a medical procedure.

### BACKGROUND

Some of typical functions of a computer-assisted surgery (CAS) system with navigation include presurgical planning of a procedure and presenting preoperative diagnostic information and images in useful formats. The CAS system presents status information about a procedure as it takes place in real time, displaying the preoperative plan along with intraoperative data. The CAS system may be used for procedures in traditional operating rooms, interventional radiology suites, mobile operating rooms or outpatient clinics. The procedure may be any medical procedure, whether surgical or non-surgical.

Surgical navigation systems are used to display the position and orientation of surgical instruments and medical implants with respect to presurgical or intraoperative medical imagery datasets of a patient. These images include pre and intraoperative images, such as two-dimensional (2D) fluoroscopic images and three-dimensional (3D) magnetic resonance imaging (MRI) or computed tomography (CT).

Navigation systems locate markers attached or fixed to an object, such as surgical instruments and patient. Most commonly these tracking systems are optical and electro-magnetic. Optical tracking systems have one or more stationary cameras that observes passive reflective markers or active infrared LEDs attached to the tracked instruments or the patient. Eye-tracking solutions are specialized optical tracking systems that measure gaze and eye motion relative to a user's head. Electro-magnetic systems have a stationary field generator that emits an electromagnetic field that is sensed by coils integrated into tracked medical tools and surgical instruments.

### SUMMARY OF THE INVENTION

Incorporating image segmentation processes that automatically identify various bone landmarks, based on their density, can increase planning accuracy. One such bone landmark is the spinal pedicle, which is made up of dense cortical bone making its identification utilizing image segmentation easier. The pedicle is used as an anchor point for various types of medical implants. Achieving proper implant placement in the pedicle is heavily dependent on the trajectory selected for implant placement. Ideal trajectory is identified by surgeon based on review of advanced imaging (e.g., CT or MRI), goals of the surgical procedure, bone density, presence or absence of deformity, anomaly, prior surgery, and other factors. The surgeon then selects the appropriate trajectory for each spinal level. Proper trajectory generally involves placing an appropriately sized implant in the center of a pedicle. Ideal trajectories are also critical for placement of inter-vertebral biomechanical devices.

Another example is placement of electrodes in the thalamus for the treatment of functional disorders, such as Parkinson's. The most important determinant of success in patients undergoing deep brain stimulation surgery is the optimal placement of the electrode. Proper trajectory is defined based on preoperative imaging (such as MRI or CT) and allows for proper electrode positioning.

Another example is minimally invasive replacement of prosthetic/biologic mitral valve in for the treatment of mitral valve disorders, such as mitral valve stenosis or regurgitation. The most important determinant of success in patients undergoing minimally invasive mitral valve surgery is the optimal placement of the three dimensional valve.

The fundamental limitation of surgical navigation systems is that they provide restricted means of communicating to the surgeon. Currently-available navigation systems present some drawbacks.

Typically, one or several computer monitors are placed at some distance away from the surgical field. They require the surgeon to focus the visual attention away from the surgical field to see the monitors across the operating room. This results in a disruption of surgical workflow. Moreover, the monitors of current navigation systems are limited to displaying multiple slices through three-dimensional diagnostic image datasets, which are difficult to interpret for complex 3D anatomy.

The fact that the screen of the surgical navigation system is located away from the region of interest (ROI) of the surgical field requires the surgeon to continuously look back and forth between the screen and the ROI. This task is not intuitive and results in a disruption to surgical workflow and decreases planning accuracy.

For example, a system of such type is disclosed in U.S. Pat. No. 9,532,848, which discloses a system for assisting a user manipulating an object during a surgery, the system comprising a tracking device for tracking the object and for generating tracking data for the object and a sterilized displaying device located in a volume within the sterile field defined as being above a plane of and delimited by an operating table and below the shoulders of an operator standing next to a patient lying on the operating table, the displaying device being supported directly by the operating table. Even though the displaying device is positioned very close to the patient being operated, the surgeon still needs to look back and forth between the screen and the ROI.

When defining and later executing an operative plan, the surgeon interacts with the navigation system via a keyboard and mouse, touchscreen, voice commands, control pendant, foot pedals, haptic devices, and tracked surgical instruments. Based on the complexity of the 3D anatomy, it can be difficult to simultaneously position and orient the instrument in the 3D surgical field only based on the information displayed on the monitors of the navigation system. Similarly, when aligning a tracked instrument with an operative plan, it is difficult to control the 3D position and orientation of the instrument with respect to the patient anatomy. This can result in an unacceptable degree of error in the preoperative plan that will translate to poor surgical outcome.

One drawback related to head-worn apparatus, such as three-dimensional display glasses is that they must provide high-resolution display to generate a signal of satisfactory quality, in particular for a large field of view. Moreover, the head-worn displays have a limited field of view. Moreover, they need very precise positioning control, as the slightest changes of surgeon's head require repositioning of the displayed image —it's quite hard to precisely and consistently keep the head-mounted display at exactly the same position and orientation with respect to the surgeon's eyes, perfect virtual/real collocation of patient anatomy is difficult to achieve. Augmented reality head-mounted devices are designed to display virtual images located at roughly the same height as the user's head, and at a certain distance from the user's body, which is not appropriate for surgery. This makes it almost impossible to achieve virtual/real anatomical superposition at a location that is reachable by the surgeon's hands. Also, the significant weight of the head-worn apparatus or head mounted display (HMD) can produce tiredness on the surgeon after a long period of usage. The surgeon has limited range of movements, it makes it harder to maneuver the head during the surgery and talk to other physicians. Moreover, use of head mounted displays involves vergence-accommodation conflict, that causes fatigue and discomfort of the user. There is disclosed herein a surgical navigation system comprising: a 3D display with a see-through mirror that is partially transparent and partially reflective; a tracking system comprising means for real-time tracking of: a surgeon's head, the see-through mirror and a patient anatomy to provide current position and orientation data; a source of a patient anatomy data; a surgical navigation image generator configured to generate a surgical navigation image comprising at least the patient anatomy data, in accordance to the current position and orientation data provided by the tracking system based on the current relative position and orientation of the surgeon's head, the see-through mirror and the patient anatomy; wherein the 3D display is configured to emit the surgical navigation image towards the see-through mirror, such that an augmented reality image collocated with the patient anatomy in the surgical field underneath the see-through mirror is visible to a viewer looking from above the see-through mirror towards the surgical field.

The 3D display may comprise a 3D projector, a plurality of opaque mirrors and a projection screen, wherein the 3D projector is configured to project the surgical navigation image towards the plurality of opaque mirrors for reflecting the surgical navigation image towards the projection screen for showing the surgical navigation image for emission towards the see-through mirror.

The 3D display may comprise a 3D monitor for showing the surgical navigation image for emission towards the see-through mirror.

The 3D display may comprise a 3D projector and a projection screen, wherein the 3D projector is configured to project the surgical navigation image towards the projection screen for showing the surgical navigation image for emission towards the see-through mirror.

The see-through mirror may be configured to be positioned, when the system is in use, at a distance (d1) from the surgeon's head which is shorter than the distance (d2) from the surgical field of the patient anatomy.

The see-through mirror may be fixed to the 3D display by means of a detachable arm.

The see-through mirror may be fixed to the 3D display by means of a foldable arm.

The see-through mirror may be disposable.

The surgical navigation image generator may be controllable by an input interface comprising at least one of: foot-operable pedals, a microphone, a joystick, an eye-tracker.

The system may further comprise an adjustable ceiling-mounted surgical boom, wherein the 3D display is attached to the adjustable ceiling-mounted surgical boom.

The system may further comprise an adjustable floor-supported structure, wherein the 3D display is attached to the adjustable floor-supported structure.

The tracking system may comprise a plurality of arranged fiducial markers, including a head array, a display array, a patient anatomy array, a surgical instrument array, as well as a fiducial marker tracker, which is configured to determine in real time the position and orientation of each of the components of the surgical navigation system.

At least one of the head array, the display array, the patient anatomy array, the instrument array may contain several fiducial markers that are not all coplanar.

The tracking system may further comprises means for real-time tracking of surgical instruments; and the system may further comprise a source of virtual surgical instrument model; and the surgical navigation image generator may be further configured to generate the surgical navigation image comprising the virtual surgical instruments based on the current relative position and orientation of the surgeon's head, the see-through mirror and the surgical instruments.

There is also disclosed herein a method for providing an augmented reality image during an operation, comprising: providing a 3D display with a see-through mirror that is partially transparent and partially reflective; providing a tracking system comprising means for real-time tracking of a surgeon's head, the see-through mirror and a patient anatomy to provide current position and orientation data; providing a source a patient anatomy data; generating, by a surgical navigation image generator, a surgical navigation image comprising the patient anatomy data, in accordance to the current position and orientation data provided by the tracking system based on the current relative position and orientation of the surgeon's head, the see-through mirror and the patient anatomy; and emitting the surgical navigation image towards a see-through mirror that is partially transparent and partially reflective, such that an augmented reality image collocated with the patient anatomy in the surgical field underneath the see-through mirror is visible to a viewer looking from above the see-through mirror towards the surgical field.

The method may further comprise providing the tracking system that further comprises means for real-time tracking of surgical instruments; providing a source of virtual surgical instrument model; and generating, by the surgical navigation image generator, the surgical navigation image comprising the virtual surgical instruments based on the current relative position and orientation of the surgeon's head, the see-through mirror and the surgical instruments.

The intended use of this invention is both presurgical planning of ideal surgical instrument trajectory and placement, and intraoperative surgical guidance, with the objective of helping to improve surgical outcomes.

A combination of a navigated probe and a computer-assisted medical system is used for interactive creation of a trajectory for positioning of a medical device. A navigated probe facilitates the positioning of the medical device. The navigated probe is part of a computer-assisted medical system consisting of a 6-degree-of-freedom (DOF) tracker (optical, electromagnetic, inertial, or any other tracking technology) and a navigated structure. The navigated structure contains a graphical user interface (GUI) for displaying patient anatomy in three dimensions (3D), as well as a virtual representation of actual implanted medical devices (IMDs) and instruments during a surgical procedure in a real time.

The surgeon can control the navigated probe by looking at its virtual representation on the GUI and lining it up to the virtual representation of the organ to achieve a proper trajectory for medical implant placement. During the planning process, a virtual instrument is displayed on a 3D display device to indicate the dynamic 3D position and orientation of the medical device. The surgeon can interact with the probe and the computer-assisted medical system by either using a 6-DOF tracking device and/or by pressing on a set of pre-programmed pedals or using other input interfaces, such as a microphone (for voice commands), a joystick, an eye-tracker (for gaze tracking).

The presented system and method solve the critical problems of typical surgical navigation systems. First, they allow the surgeon to focus the visual attention to the surgical field by superimposing 3D patient anatomy, surgical guidance, and orthogonal planes directly onto the area of patient anatomy where the surgery is performed, without requiring the surgeon to look away from the surgical field. Secondly, they provide the surgeon a more intuitive mechanism to define and execute an operative plan by simply handling the surgical instruments in a 3D workspace that perfectly matches the operative field, without requiring the surgeon to perform a disorienting mental mapping of the information displayed by the navigation system to the 3D position and orientation of the surgical instruments with respect to the complex 3D anatomy. Moreover, by using the presented system and method, the time of operation can be reduced, as the more intuitive communication means do not distract the surgeon and do not require additional time to look away from the ROI. The system and method also does not obstruct the freedom of instrument and hand movement during the operating procedure in certain embodiments.

These and other features, aspects and advantages of the invention will become better understood with reference to the following drawings, descriptions and claims.

### BRIEF DESCRIPTION OF FIGURES

The surgical navigation system and method are presented herein by means of non-limiting example embodiments shown in a drawing, wherein:

FIG. 1A shows a layout of a surgical room employing the surgical navigation system in accordance with an embodiment of the invention;

FIG. 1B shows a layout of a surgical room employing the surgical navigation system in accordance with an embodiment of the invention;

FIG. 1C shows a layout of a surgical room employing the surgical navigation system in accordance with an embodiment of the invention;

FIG. 2A shows components of the surgical navigation system in accordance with an embodiment of the invention;

FIG. 2B shows components of the surgical navigation system in accordance with an embodiment of the invention;

FIG. 3A shows an example of an augmented reality display in accordance with an embodiment of the invention;

FIG. 3B shows an example of an augmented reality display in accordance with an embodiment of the invention;

FIG. 3C shows an example of an augmented reality display in accordance with an embodiment of the invention;

FIG. 3D shows an example of an augmented reality display in accordance with an embodiment of the invention;

FIG. 3E shows an example of an augmented reality display in accordance with an embodiment of the invention;

FIG. 4A shows an embodiment of a 3D display system comprising a projector.

FIG. 4B shows an embodiment of a 3D display system comprising a projector.

FIG. 5A shows eye tracking in accordance with an embodiment of the invention.

FIG. 5B shows eye tracking in accordance with an embodiment of the invention.

FIG. 6A shows a foldable see-through mirror in accordance with an embodiment of the invention.

FIG. 6B shows a foldable see-through mirror in accordance with an embodiment of the invention.

FIG. 6C shows a foldable see-through mirror in accordance with an embodiment of the invention.

FIG. 6D shows a foldable see-through mirror in accordance with an embodiment of the invention.

FIG. 6E show a foldable see-through mirror in accordance with an embodiment of the invention.

### DETAILED DESCRIPTION

The following detailed description is of the best currently contemplated modes of carrying out the invention. The description is not to be taken in a limiting sense, but is made merely for the purpose of illustrating the general principles of the invention.

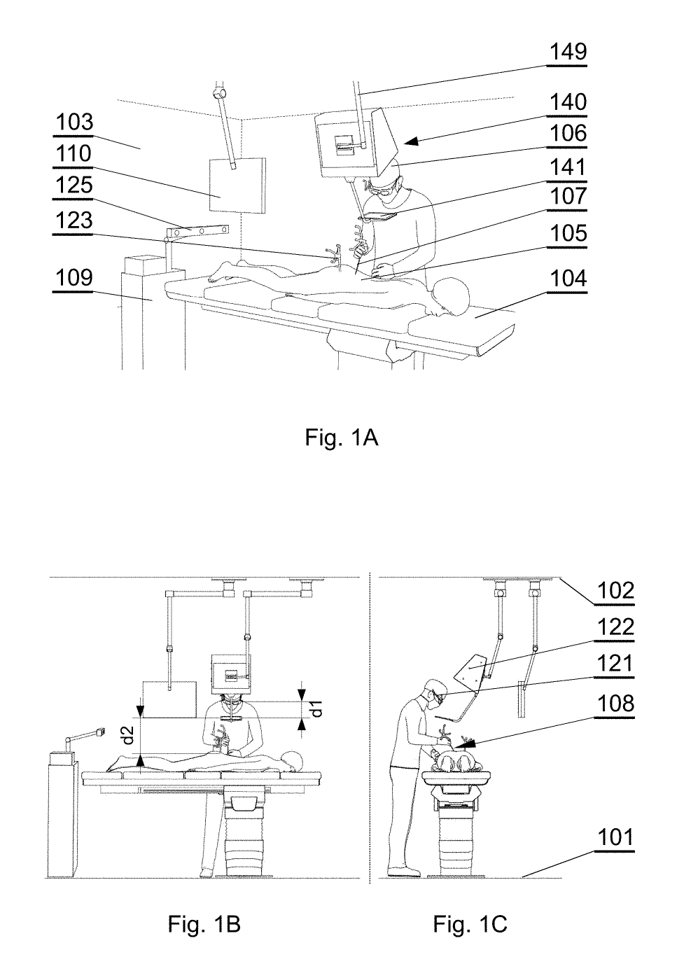

The system presented herein comprises a 3D display system 140 to be implemented directly on real surgical applications in a surgical room as shown in FIGS. 1A-1C. The 3D display system 140 comprises a 3D display 142 for emitting a surgical navigation image 142A towards a see-through mirror 141 that is partially transparent and partially reflective, such that an augmented reality image 141A collocated with the patient anatomy in the surgical field 108 underneath the see-through mirror 141 is visible to a viewer looking from above the see-through mirror 141 towards the surgical field 108.

The surgical room typically comprises a floor 101 on which an operating table 104 is positioned. A patient 105 lies on the operating table 104 while being operated by a surgeon 106 with the use of various surgical instruments 107. The surgical navigation system as described in details below can have its components, in particular the 3D display system 140, mounted to a ceiling 102, or alternatively to the floor 101 or a side wall 103 of the operating room. Furthermore, the components, in particular the 3D display system 140, can be mounted to an adjustable and/or movable floor-supported structure (such as a tripod). Components other than the 3D display system 140, such as the surgical image generator 131, can be implemented in a dedicated computing device 109, such as a stand-alone PC computer, which may have its own input controllers and display(s) 110.

In general, the system is designed for use in such a configuration wherein the distance d1 between the surgeon's eyes and the see-through mirror 141, is shorter than the distance d2, between the see-through mirror 141 and the operative field at the patient anatomy 105 being operated.

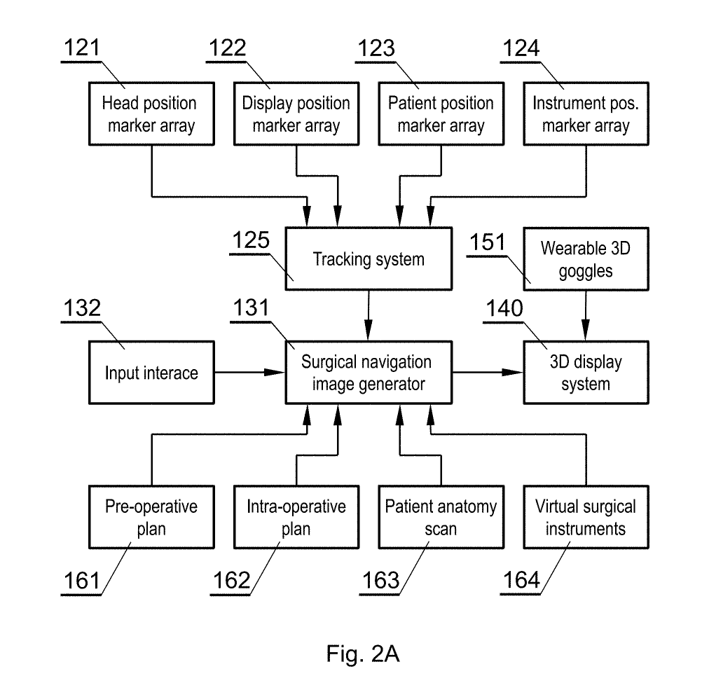

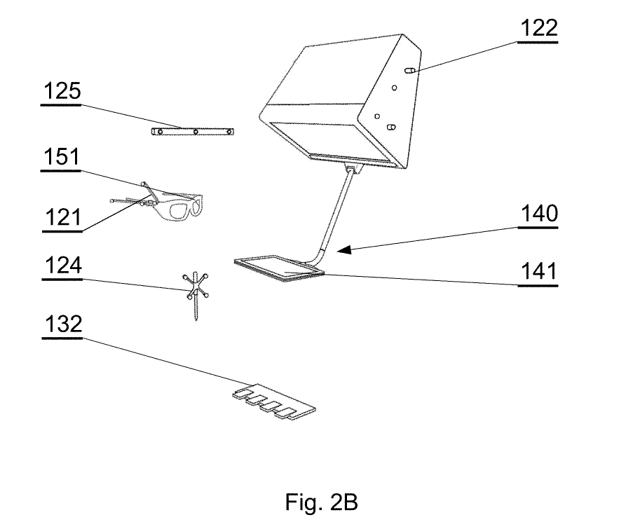

FIG. 2A shows a functional schematic presenting connections between the components of the surgical navigation system and FIG. 2B shows examples of physical embodiments of various components.

The surgical navigation system comprises a tracking system for tracking in real time the position and/or orientation of various entities to provide current position and/or orientation data. For example, the system may comprise a plurality of arranged fiducial markers, which are trackable by a fiducial marker tracker 125. Any known type of tracking system can be used, for example in case of a marker tracking system, 4-point marker arrays are tracked by a three-camera sensor to provide movement along six degrees of freedom. A head position marker array 121 can be attached to the surgeon's head for tracking of the position and orientation of the surgeon and the direction of gaze of the surgeon—for example, the head position marker array 121 can be integrated with the wearable 3D glasses 151 or can be attached to a strip worn over surgeon's head.

A display marker array 122 can be attached to the see-through mirror 141 of the 3D display system 140 for tracking its position and orientation, as the see-through mirror 141 is movable and can be placed according to the current needs of the operative setup.

A patient anatomy marker array 123 can be attached at a particular position and orientation of the anatomy of the patient.

A surgical instrument marker array 124 can be attached to the instrument whose position and orientation shall be tracked.

Preferably, the markers in at least one of the marker arrays 121-124 are not coplanar, which helps to improve the accuracy of the tracking system.

Therefore, the tracking system comprises means for real-time tracking of the position and orientation of at least one of: a surgeon's head 106, a 3D display 142, a patient anatomy 105, and surgical instruments 107. Preferably, all of these elements are tracked.

A surgical navigation image generator 131 is configured to generate an image to be viewed via the see-through mirror 141 of the 3D display system. It generates a surgical navigation image 142A comprising at least the patient anatomy data 163 (which can be generated before the operation or live during the operation), in accordance to the current position and orientation data provided by the tracking system (125) based on the current relative position and orientation of the surgeon's head (106), the see-through mirror (141) and the patient anatomy (105). Further, the surgical navigation image 142A may comprise data of the pre-operative plan 161 (which are generated and stored in a database before the operation), data of the intra-operative plan 162 (which can be generated live during the operation) and/or virtual images 164 of surgical instruments used during the operation (which are stored as 3D models in a database).

The surgical navigation image generator 131, as well as other components of the system, can be controlled by a user (i.e. a surgeon or support staff) by one or more user interfaces 132, such as foot-operable pedals (which are convenient to be operated by the surgeon), a keyboard, a mouse, a joystick, a button, a switch, an audio interface (such as a microphone), a gesture interface, a gaze detecting interface etc. The input interface(s) are for inputting instructions and/or commands.

All system components are controlled by one or more computer which is controlled by an operating system and one or more software applications. The computer may be equipped with a suitable memory which may store computer program or programs executed by the computer in order to execute steps of the methods utilized in the system. Computer programs are preferably stored on a non-transitory medium. An example of a non-transitory medium is a non-volatile memory, for example a flash memory while an example of a volatile memory is RAM. The computer instructions are executed by a processor. These memories are exemplary recording media for storing computer programs comprising computer-executable instructions performing all the steps of the computer-implemented method according the technical concept presented herein. The computer(s) can be placed within the operating room or outside the operating room. Communication between the computers and the components of the system may be performed by wire or wirelessly, according to known communication means.

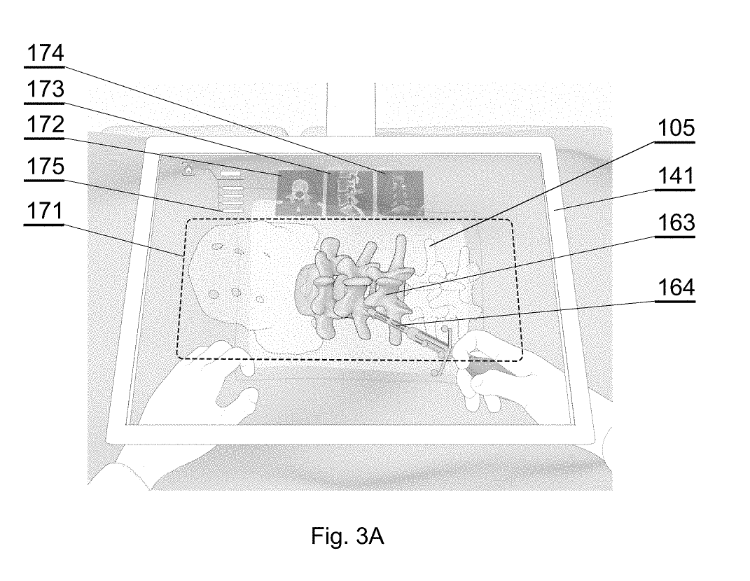

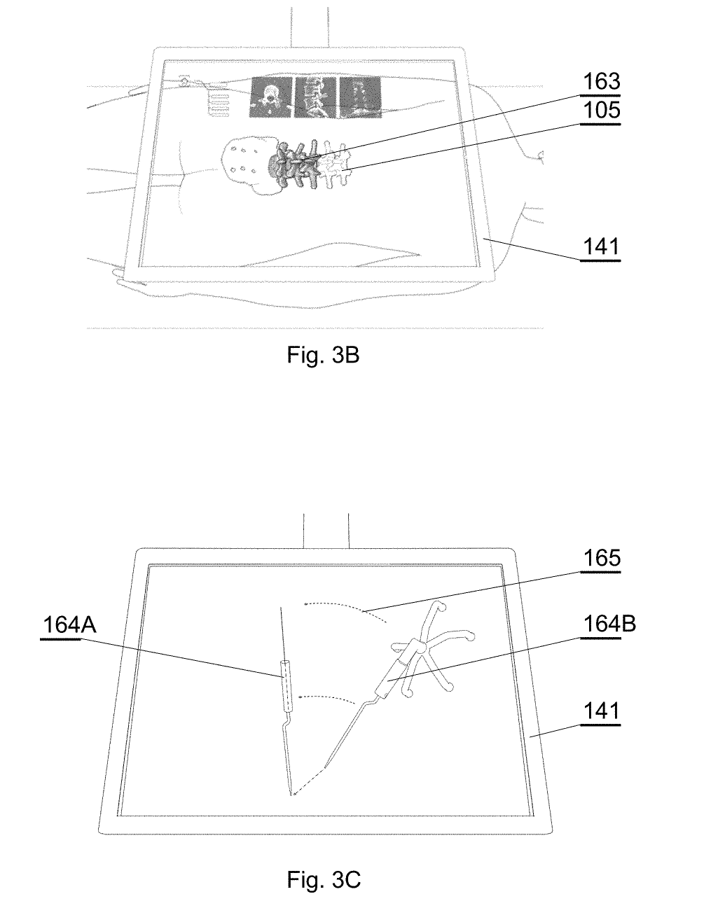

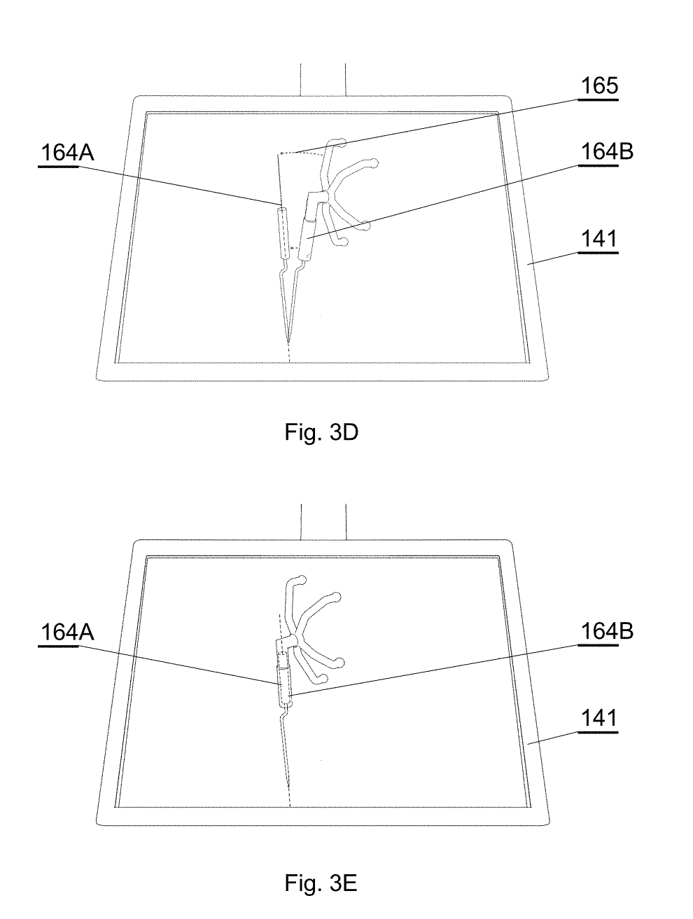

The aim of the system is to generate, via the see-through mirror 141, an augmented reality image such as shown in examples of FIGS. 3A-3E. When the surgeon looks via the see-through mirror 141, the surgeon sees the augmented reality image 141A which comprises:
the real world image: the patient anatomy, surgeon's hands and the instrument currently in use (which may be partially inserted into the patient's body and hidden under the skin);
and a computer-generated surgical navigation image 142A comprising:
   a 3D image 171 representing at least the virtual image of the patient anatomy 163, and possibly also the virtual image of the instrument 164 or surgical guidance indicating ideal trajectory and placement of surgical instruments 107, according to the pre-operative plans 161 (as shown in FIG. 3C);
   preferably, three different orthogonal planes of the patient anatomy data 163: coronal 174, sagittal 173, axial 172;
   preferably, a menu 175 for controlling the system operation.

If the 3D display 142 is stereoscopic, the surgeon shall use a pair of 3D glasses 151 to view the augmented reality image 141A. However, if the 3D display 142 is autostereoscopic, it may be not necessary for the surgeon to use the 3D glasses 151 to view the augmented reality image 141A.

Preferably, the images of the orthogonal planes 172, 173, 174 are displayed in an area next (preferably, above) to the area of the 3D image 171, as shown in FIG. 3A, wherein the 3D image 171 occupies more than 50% of the area of the see-through mirror 141.

The location of the images of the orthogonal planes 172, 173, 174 may be adjusted in real time depending on the location of the 3D image 171, when the surgeon changes the position of the head during operation, such as not to interfere with the 3D image 171.

The surgical navigation image 142A is generated by the image generator 131 in accordance with the tracking data provided by the fiducial marker tracker 125, in order to superimpose the anatomy images and the instrument images exactly over the real objects, in accordance with the position and orientation of the surgeon's head. The markers are tracked in real time and the image is generated in real time. Therefore, the surgical navigation image generator 131 provides graphics rendering of the virtual objects (patient anatomy, surgical plan and instruments) collocated to the real objects according to the perspective of the surgeon's perspective.

For example, surgical guidance may relate to suggestions (virtual guidance clues 164) for placement of a pedicle screw in spine surgery or the ideal orientation of an acetabular component in hip arthroplasty surgery. These suggestions may take a form of animations that show the surgeon whether the placement is correct. The suggestions may be displayed both on the 3D holographic display and the orthogonal planes. The surgeon may use the system to plan these orientations before or during the surgical procedure.

In particular, the 3D image 171 is adapted in real time to the position and orientation of the surgeon's head. The display of the different orthogonal planes 172, 173, 174 may be adapted according to the current position and orientation of the surgical instruments used.

The aligning the line of sight of the surgeon onto the see-through mirror with the patient anatomy underneath the see-through mirror, involving the scaling and orientation of the image, can be realized based on known solutions in the field of computer graphics processing, in particular for virtual reality, including virtual scene generation, using well-known mathematical formulas and algorithms related to viewer centered perspective. For example, such solutions are known from various tutorials and textbooks (such as “The Future of the CAVE” by T. A. DeFanti et al, Central European Journal of Engineering, 2010, DOI: 10.2478/s13531-010-0002-5).

FIG. 3B shows an example indicating collocation of the virtual image of the patient anatomy 163 and the real anatomy 105.

For example, as shown in FIG. 3C, the 3D image 171 may demonstrate a mismatch between a supposed position of the instrument according to the pre-operative plan 161, displayed as a first virtual image of the instrument 164A located at its supposed position, and an actual position of the instrument, visible either as the real instrument via the see-through mirror and/or a second virtual image of the instrument 164B overlaid on the current position of the instrument. Additionally, graphical guiding cues, such as arrows 165 indicating the direction of the supposed change of position, can be displayed.

FIG. 3D shows a situation wherein the tip of the supposed position of the instrument displayed as the first virtual image 164A according to the pre-operative plan 161 matches the tip of the real surgical instrument visible or displayed as the second virtual image 164B. However, the remainder of objects do not match, therefore the graphical cues 165 still indicate the need to change its position. The surgical instrument is close to the correct position and the system may provide information on how close the surgical instrument is to the planned position.

FIG. 3E shows a situation wherein the supposed position of the real surgical instrument matches the position of the instrument according to the pre-operative plan 161, i.e. the correct position for surgery. In this situation the graphical cues 165 are no longer displayed, but the virtual images 164A, 164B may be changed to indicate the correct position, e.g. by highlighting it or blinking.

The see-through mirror (also called a half-silvered mirror) 141 is at least partially transparent and partially reflective, such that the viewer can see the real world behind the screen but the screen also reflects the surgical navigation image generated by the display apparatus located above it.

For example, a see-through mirror as commonly used in teleprompters can be used. For example, the see-through mirror 141 can have a reflective and transparent rate of 50R/50T, but other rates can be used as well.

The surgical navigation image is emitted from above the see-through mirror 141 by the 3D display 142.

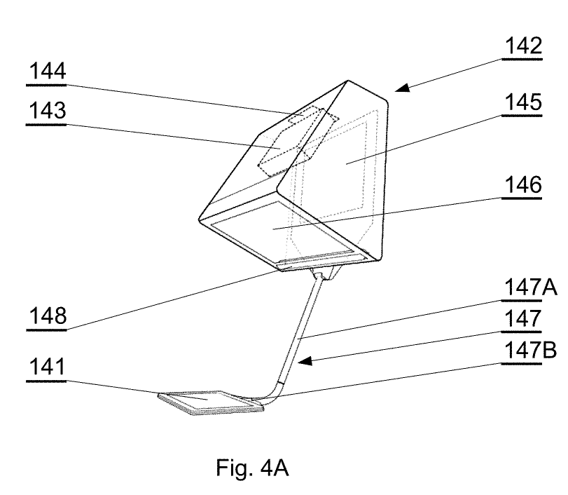

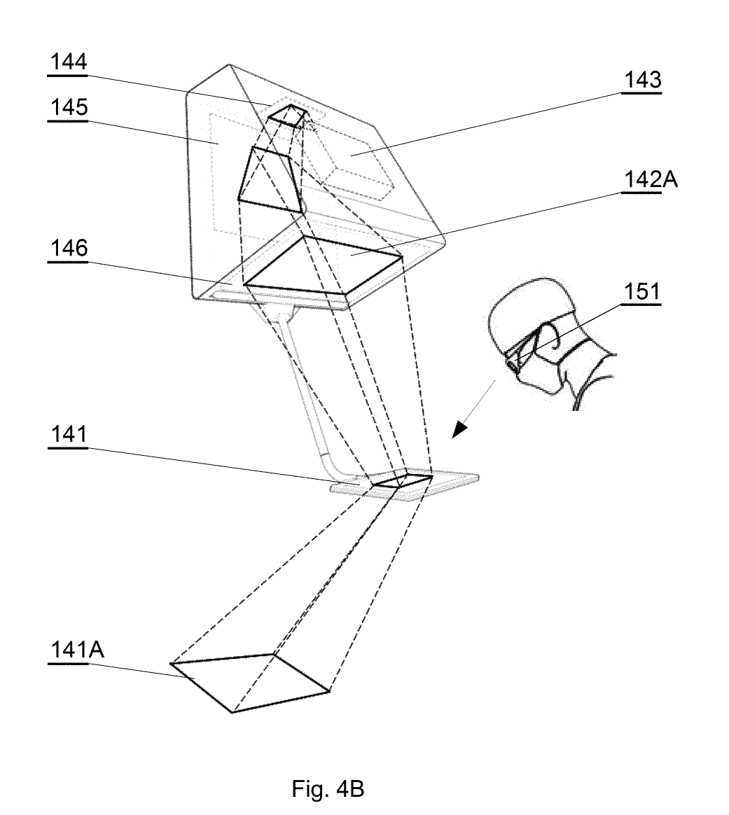

In an example embodiment as shown in FIG. 4A, a special design of the 3D display 142 is provided that is compact in size to facilitate its mounting within a limited space at the operating room. That design allows generating images of relatively large size, taking into account the small distance between the 3D display 142 and the see-through mirror 141, without the need to use wide-angle lens that could distort the image.

The 3D display 142 comprises a 3D projector 143, such as a DLP projector, that is configured to generate an image, as shown in FIG. 4B (by the dashed lines showing image projection and solid lines showing images generated on particular reflective planes). The image from the 3D projector 143 is firstly refracted by an opaque top mirror 144, then it is refracted by an opaque vertical mirror 145 and subsequently placed on the correct dimensions on a projection screen 146 (which can be simply a glass panel). The projection screen 146 works as a rear-projection screen or a small bright 3D display. The image displayed at the projection screen 146 is reflected by the see-through mirror 141 which works as an augmented reality visor. Such configuration of the mirrors 144, 145 allows the image generated by the 3D projector 143 to be shown with an appropriate size at the projection screen 146. The fact that the projection screen 146 emits an enlarged image generated by the 3D projector 143 makes the emitted surgical navigation image bright, and therefore well visible when reflected at the see-through mirror 141. Reference 141A indicates the augmented reality image as perceived by the surgeon when looking at the see-through mirror 141.

Alternatively, a projector of a wider angle than the source projector of FIG. 4A can be used as the 3D projector 143, wherein the wide angle projector is emitting the surgical navigation image directly towards the projection screen 146 of the embodiment described in FIG. 4A.

Alternatively, a 3D monitor can be used directly in place of the projection screen 146.

The see-through mirror 141 is held at a predefined position with respect to the 3D projector 143, in particular with respect to the 3D projector 143, by an arm 147.

In one embodiment, as shown in FIG. 4A, the arm 147 may have a first portion 147A fixed to the casing of the 3D display 142 and a second portion 147B detachably fixed to the first portion 147A. The first portion 147A may have a protective sleeve overlaid on it. The second portion 147B, together with the see-through mirror 141, may be disposable in order to keep sterility of the operating room, as it is relatively close to the operating field and may be contaminated during the operation. The arm can also be foldable upwards to leave free space of the work space when the arm and augmented reality are not needed

If a need arises to adapt the position of the augmented reality screen with respect to the surgeon's head (for example, to accommodate the position depending on the height of the particular surgeon), the position of the whole 3D display system 140 can be changed, for example by manipulating an adjustable holder (a surgical boom) 149 on FIG. 1A, by which the 3D display 142 is attachable to an operating room structure, such as a ceiling, a wall or a floor.

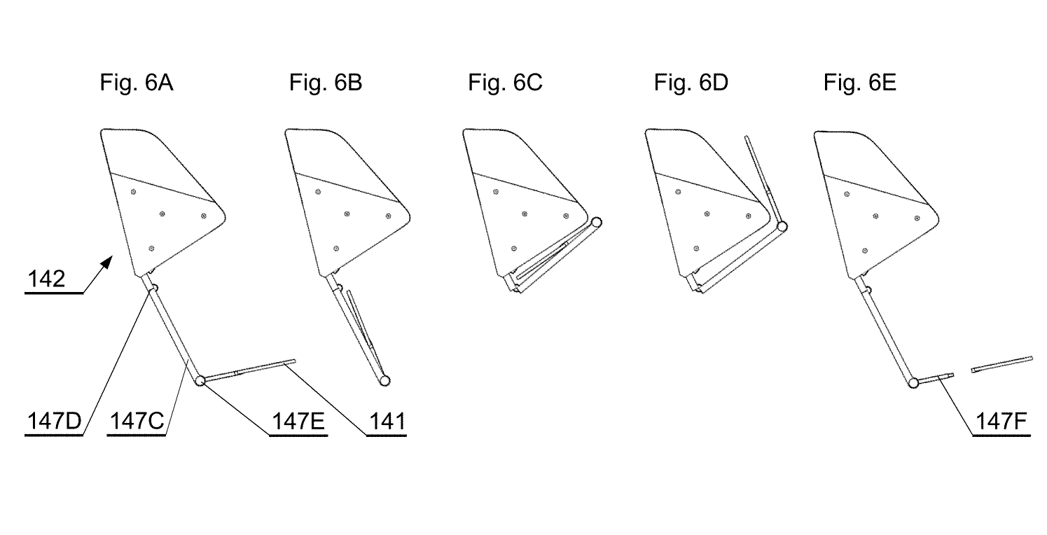

In another embodiment, as shown in FIGS. 6A-6E, the arm 147 may comprise a bar 147C attached at a first end via a first pivot joint 147D to the body of the 3D display and having a second end with a second pivot joint 147E to which the see-through mirror 141 is attached. Therefore, the see-through mirror 141 can be arranged in various positions that do not obstruct the field of view of the surgeon, such as shown in FIGS. 6B, 6C and 6D. As further shown in FIG. 6E, the see-through mirror can be detachably attached to a bar 147F attached to the second pivot joint 147E.

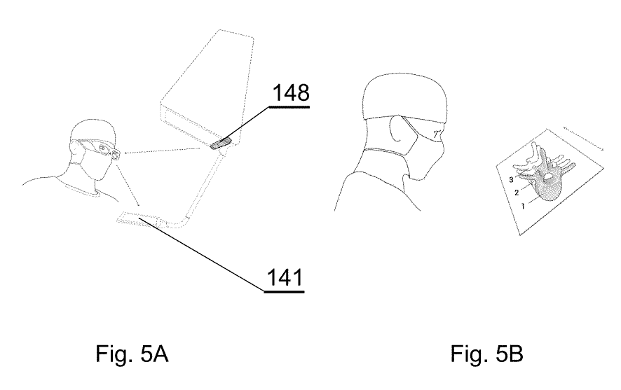

An eye tracker 148 module can be installed at the casing of the 3D display 142 or at the see-through mirror 141 or at the wearable glasses 151, to track the position and orientation of the eyes of the surgeon and input that as commands via the gaze input interface to control the display parameters at the surgical navigation image generator 131, for example to activate different functions based on the location that is being looked at, as shown in FIGS. 5A and 5B.

For example, the eye tracker 148 may use infrared light to illuminate the eyes of the user without affecting the visibility of the user, wherein the reflection and refraction of the patterns on the eyes are utilized to determine the gaze vector (i.e. the direction at which the eye is pointing out). The gaze vector along with the position and orientation of the user's head is used to interact with the graphical user interface. However, other eye tracking algorithms techniques can be used as well.

It is particularly useful to use the eye tracker 148 along with the pedals 132 as the input interface, wherein the surgeon may navigate the system by moving a cursor by sight and inputting commands (such as select or cancel) by pedals.

While the invention has been described with respect to a limited number of embodiments, it will be appreciated that many variations, modifications and other applications of the invention may be made. Therefore, the claimed invention as recited in the claims that follow is not limited to the embodiments described herein.

What is claimed is:

1. A surgical navigation system comprising:
a 3D display with a see-through mirror that is partially transparent and partially reflective;
a tracking system comprising means for real-time tracking of: a surgeon's head, the see-through mirror and a patient anatomy to provide current position and orientation data;
a source of a patient anatomy data;
a surgical navigation image generator configured to generate a surgical navigation image comprising at least the patient anatomy data, in accordance to the current position and orientation data provided by the tracking system based on the current relative position and orientation of the surgeon's head, the see-through mirror and the patient anatomy;
wherein the 3D display is configured to emit the surgical navigation image towards the see-through mirror, such that an augmented reality image collocated with the patient anatomy in the surgical field underneath the see-through mirror is visible to a viewer looking from above the see-through mirror towards the surgical field.

2. The system of claim 1, wherein the 3D display comprises a 3D projector, a plurality of opaque mirrors and a projection screen, wherein the 3D projector is configured to project the surgical navigation image towards the plurality of opaque mirrors for reflecting the surgical navigation image towards the projection screen for showing the surgical navigation image for emission towards the see-through mirror.

3. The system of claim 1, wherein the 3D display comprises a 3D monitor for showing the surgical navigation image for emission towards the see-through mirror.

4. The system of claim 1, wherein the 3D display comprises a 3D projector and a projection screen, wherein the 3D projector is configured to project the surgical navigation image towards the projection screen for showing the surgical navigation image for emission towards the see-through mirror.

5. The system of claim 1, wherein the see-through mirror is configured to be positioned, when the system is in use, at a distance (d1) from the surgeon's head which is shorter than the distance (d2) from the surgical field of the patient anatomy.

6. The system of claim 1, wherein the see-through mirror is fixed to the 3D display by means of a detachable arm.

7. The system of claim 1, wherein the see-through mirror is fixed to the 3D display by means of a foldable arm.

8. The system of claim 1, wherein the see-through mirror is disposable.

9. The system of claim 1, wherein the surgical navigation image generator is controllable by an input interface comprising at least one of: foot-operable pedals, a microphone, a joystick, an eye-tracker.

10. The system of claim 1, further comprising an adjustable ceiling-mounted surgical boom, wherein the 3D display is attached to the adjustable ceiling-mounted surgical boom.

11. The system of claim 1, further comprising an adjustable floor-supported structure, wherein the 3D display is attached to the adjustable floor-supported structure.

12. The system of claim 1, wherein the tracking system comprises plurality of arranged fiducial markers, including a head array, a display array, a patient anatomy array, a surgical instrument array, as well as a fiducial marker tracker, which is configured to determine in real time the position and orientation of each of the components of the surgical navigation system.

13. The system of claim 12, wherein at least one of the head array, the display array, the patient anatomy array, the instrument array contains several fiducial markers that are not all coplanar.

14. The system of claim 1:
wherein the tracking system further comprises means for real-time tracking of surgical instruments;
wherein the system further comprises a source of virtual surgical instrument model;
and the surgical navigation image generator is further configured to generate the surgical navigation image comprising the virtual surgical instruments based on the current relative position and orientation of the surgeon's head, the see-through mirror and the surgical instruments.

15. A method for providing an augmented reality image during an operation, comprising:
providing a 3D display with a see-through mirror that is partially transparent and partially reflective;
providing a tracking system comprising means for real-time tracking of a surgeon's head, the see-through mirror and a patient anatomy to provide current position and orientation data;
providing a source a patient anatomy data;
generating, by a surgical navigation image generator, a surgical navigation image comprising the patient anatomy data, in accordance to the current position and orientation data provided by the tracking system based on the current relative position and orientation of the surgeon's head, the see-through mirror and the patient anatomy; and
emitting the surgical navigation image towards a see-through mirror that is partially transparent and partially reflective, such that an augmented reality image collocated with the patient anatomy in the surgical field underneath the see-through mirror is visible to a viewer looking from above the see-through mirror towards the surgical field.

16. The method of claim 15, further comprising:
providing the tracking system that further comprises means for real-time tracking of surgical instruments;
providing a source of virtual surgical instrument model; and
generating, by the surgical navigation image generator, the surgical navigation image comprising the virtual surgical instruments based on the current relative position and orientation of the surgeon's head, the see-through mirror and the surgical instruments.
